(19) United States
(12) Patent Application Publication
(10) Pub. No.: US 2014/0330079 A1
(43) Pub. Date: Nov. 6, 2014

(54) LIVING BODY INTRODUCTION APPARATUS

(71) Applicant: OLYMPUS MEDICAL SYSTEMS CORP., Tokyo (JP)

(72) Inventors: Ryosuke ISHIZAKI, Tama-shi (JP); Takehiro NISHIIE, Akishima-shi (JP)

(73) Assignee: OLYMPUS MEDICAL SYSTEMS CORP., Tokyo (JP)

(21) Appl. No.: 14/275,987

(22) Filed: May 13, 2014

Related U.S. Application Data
(63) Continuation of application No. PCT/JP2013/059248, filed on Mar. 28, 2013.

(30) Foreign Application Priority Data
Mar. 30, 2012 (JP) 2012-081812

(51) Int. Cl. A61B 1/00

(52) U.S. Cl. CPC A61B 1/00133
USPC 600/114

(57) ABSTRACT

In a living body introduction apparatus, an inner rotating cylinder is covered with a stretchable cover having an inner circumferential length shorter than an outer circumferential length of the inner rotating cylinder. A portion of the cover pushed out in a protruding form by a roller disposed on an outer circumferential surface of the inner rotating cylinder enters a circular groove formed inside an outer rotating cylinder. Rotation of the inner rotating cylinder circularly moves the protruding portion on the outer circumferential surface. The protruding cover portion pushes the circular groove to rotate the outer rotating cylinder.

This is a Continuation application of PCT Application No. PCT/JP2013/059248, filed Mar. 28, 2013, which was published under PCT Article 21(2) in Japanese. This application is based upon and claims the benefit of priority from prior Japanese Patent Application No. 2012-081812, filed Mar. 30, 2012, the entire contents of which are incorporated herein by reference.

### BACKGROUND OF THE INVENTION

1. Field of the Invention

The present invention relates to a living body introduction apparatus provided on a leading end side of an insertion portion.

2. Description of the Related Art

Endoscope apparatuses are generally known which comprise an insertion section inserted into a lumen to allow observation of images picked up using an image pickup section provided at a leading end portion of the insertion section. For example, U.S. Patent Application No. US2012/0029281A1 proposes an endoscope apparatus with a living body introduction apparatus which is placed after a leading end of an insertion section or a bending section at the leading end is placed and which rotates around a longitudinal axis.

The living body introduction apparatus comprises a fin provided on a surface of an outer rotating cylinder of the insertion section and comprising a soft material such as rubber or resin, the fin being formed to wrap spirally around the surface. The fin is rotated while being inserted through the lumen to perform an auxiliary operation for insertion of the insertion section by advancing while pushing the inner wall, fold, and the like of the lumen away backward.

The living body introduction apparatus draws a rotational driving force exerted by a motor provided in a manipulation section of the endoscope and serving as a driving source, into the insertion section through a wire or the like to rotate a driving gear provided at a leading end of a drive shaft. The gear allows an inner rotating cylinder to be fitted over an outer circumference of the insertion section. The rotating cylinder comprises gear teeth annularly provided along an inner circumferential surface of the rotating cylinder. The rotating cylinder fit on the outer circumferential surface of the insertion section.

Moreover, plurality of rollers is arranged on an upper surface of the inner rotating cylinder so as to be rotatable in position in a longitudinal direction of the insertion section. The rollers are arranged in alignment with recesses (roller spacings) formed by a pair of small rollers provided on an inner surface side of the outer rotating cylinder, to transmit a rotating force of the inner rotating cylinder to the outer rotating cylinder. Thus, the outer rotating cylinder rotates integrally with the inner rotating cylinder in conjunction with rotation of the inner rotating cylinder.

The above-described living body introduction apparatus is inserted into the lumen in a living body or the like for use and thus needs to have a liquid-tight structure. That is, the liquid-tight structure is needed in order to prevent structural sites from being corroded as a result of inward infiltration of a liquid such as a body fluid and to ensure a sufficient sterilization effect.

In a rotating mechanism comprising the inner rotating cylinder and the outer rotating cylinder and utilizing the plurality of rollers, the inner rotating cylinder including the rollers is covered with a cover comprising a tube-like waterproof sheet. The cover allows openings at opposite ends of the cover to be fixed in a liquid-tight manner to non-rotating insertion sections (longitudinal axis direction sides of the insertion section) that support the inner rotating cylinder so that the inner rotating cylinder is rotatable. This configuration allows the rollers to rotate on the inner rotating cylinder while pushing the cover up so that the cover forms protruding portions when the inner rotating cylinder rotates inside the cover. Each of the protruding portions is fitted into the recess between the small rollers pairs spaced from one another on the inner surface side of the outer rotating cylinder. Thus, pushing the small roller pairs ahead rotates the outer rotating cylinder.

### BRIEF SUMMARY OF THE INVENTION

According to an embodiment of the present invention, there is provided an introduction apparatus comprising: an insertion section configured to extend along a longitudinal axis and to be able to be inserted into a lumen; a driving force generating section generating a rotational driving force; an inner rotating cylinder provided the projecting portions comprising on an outer circumferential surface and formed shaped like a cylinder and, in the insertion section at any position on the insertion section and configured to be rotatable around the longitudinal axis, the inner rotating cylinder rotating in accordance with a rotational driving force from the rotational driving source; a covering member configured to have an inner circumferential length equal to or smaller than an outer circumferential length of the inner rotating cylinder includings the projecting portions, the covering member annularly covering the inner rotating cylinder including the rotating site provided on the outer circumferential surface of the inner rotating cylinder, in such a manner as to tightly contact the inner rotating cylinder includings the projecting portions, the covering member fixing opening ends thereof at opposite ends thereof to non-rotating sites located beyond the inner rotating cylinder; an outer rotating cylinder having an engagement section formed on an inner circumferential side thereof and which are engageable with the projecting portions covered with the covering member, the outer rotating cylinder being fitted on a casing side of the inner rotating cylinder covered with the covering member, the engagement section being pushed ahead and rotated by a protruding portion of the covering member pushed up by the projecting portions as a result of rotation of the inner rotating cylinder; and an introduction propulsion unit fitted to turn with over the outer rotating cylinder in such a manner as to cover an outer surface of the outer rotating cylinder and comprising a spiral propulsion site on an outer surface of the introduction propulsion unit, the introduction propulsion unit supporting rotational introduction into the lumen.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute apart of the specification, illustrate embodiments of the invention, and together with the general description given above and the detailed description of the embodiments given below, serve to explain the principles of the invention.

FIG. 1 is a conceptual diagram showing an external configuration of an endoscope apparatus with a living body introduction apparatus on an insertion section according to a first embodiment.

FIG. 2 is a diagram showing a configuration of the living body introduction apparatus in section.

FIG. 3A is a cross-sectional view showing the configuration and taken along A-A in FIG. 2.

FIG. 3B is a simplified cross-sectional view taken along A-A in FIG. 2.

FIG. 4A is a diagram illustrating an assembly structure of the living body introduction apparatus.

FIG. 4B is a diagram continued from FIG. 4A and illustrating the assembly structure of the living body introduction apparatus.

FIG. 4C is a diagram continued from FIG. 4B and illustrating the assembly structure of the living body introduction apparatus.

FIG. 4D is a diagram continued from FIG. 4C and illustrating the assembly structure of the living body introduction apparatus.

FIG. 5A is a diagram continued from FIG. 4D and illustrating the assembly structure of the living body introduction apparatus.

FIG. 5B is a diagram continued from FIG. 5A and illustrating the assembly structure of the living body introduction apparatus.

FIG. 5C is a diagram continued from FIG. 5B and illustrating the assembly structure of the living body introduction apparatus.

FIG. 5D is a diagram continued from FIG. 5C and illustrating the assembly structure of the living body introduction apparatus.

FIG. 6 is a diagram showing a configuration of a living body introduction apparatus according to a second embodiment in section0.

FIG. 7 is a cross-sectional view showing the configuration and taken along B-B in FIG. 6.

FIG. 8 is a diagram showing an external configuration of an outer rotating cylinder configured integrally with a spiral tube (a casing) of the living body introduction apparatus.

FIG. 9A is a diagram showing, in section, the configuration in which a cover is slackened or creased.

FIG. 9B is a diagram showing, in section, the configuration in which the cover is slackened or creased.

FIG. 9C is a diagram showing the sideways appearance of the outer rotating cylinder with the cover slackened or creased.

### DETAILED DESCRIPTION OF THE PREFERRED EMBODIMENTS

Embodiments of the present invention will be described below in detail with reference to the drawings.

### Embodiment 1

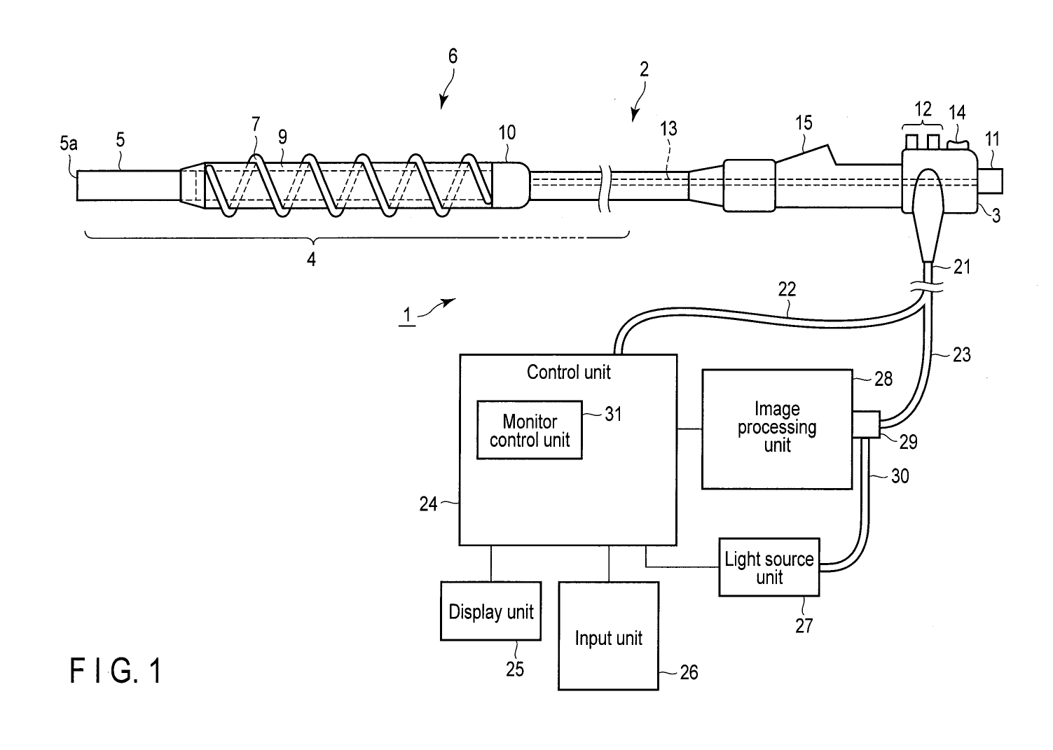

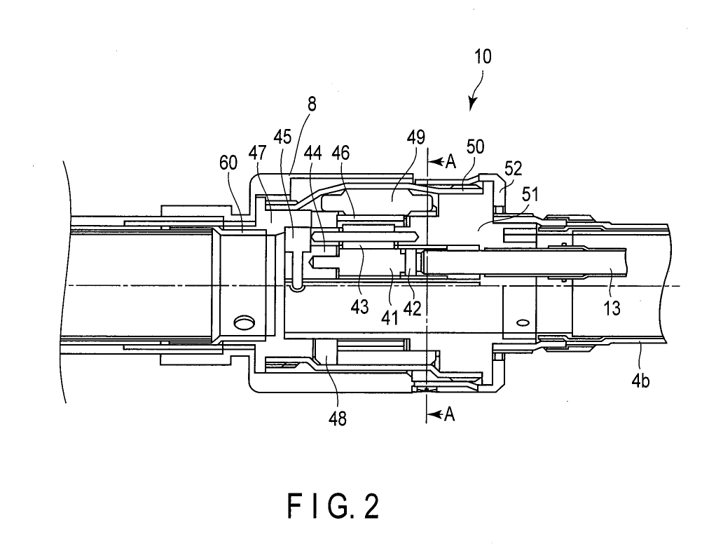

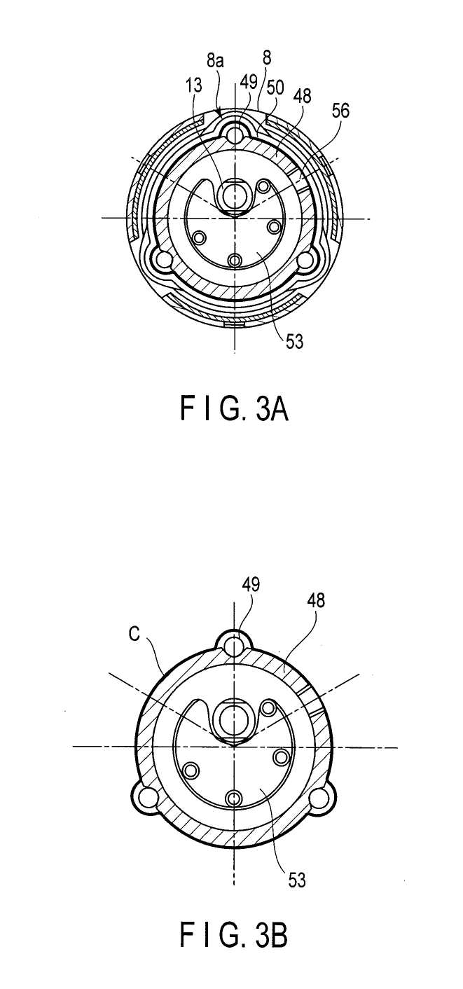

FIG. 1 is a conceptual diagram showing an external configuration of an endoscope apparatus with an insertion section on which a living body introduction apparatus according to a first embodiment is provided. FIG. 2 is a diagram showing a configuration of the living body introduction apparatus in section. FIG. 3A is a cross-sectional view showing the configuration and taken along A-A in FIG. 2.

An endoscope apparatus 1 according to the present embodiment comprises an insertion section 2, a manipulation section 3 provided on a base end side of the insertion section 2, and a driving unit system.

The insertion section 2 comprises a bending section 5 formed to conform with bending of a lumen. The bending section 5 comprises an image pickup section, awash nozzle, and a channel hole all formed at a leading end portion 5a of the bending section 5; a pair of forceps or the like is inserted through the channel hole. A living body introduction apparatus 6 described below is provided at a position backward of the leading end of the insertion section 2 at any distance from the leading end. A member insertion section 15 with an opening end of the channel formed therein is disposed on a base end side of the insertion section 2 and between the insertion section 2 and the manipulation section 3.

A knob and a switch 12 allowing various manipulations including a bending manipulation to be performed are disposed on the manipulation section 3. A motor 11 described below is disposed opposite the base end side of the insertion section 2, and an operation switch 14 for driving the motor is disposed in the vicinity of the motor 11. The motor 11 may be contained in the main body of the manipulation section 3.

The driving unit system comprises a control section 24 that controls the whole apparatus including units, an image processing unit 28 that processes images picked up using the image pickup section, a light source unit 27 that emits illumination light illuminating an image pickup target, an input unit 26 that inputs instructions and the like to the control section 24, and a display unit 25 that displays picked-up images. In the description below, electric or optical connections using connectors are referred to as connector connections.

The image processing unit 28 is connector-connected to the manipulation section 3 through a universal cable 23 (integrated cable 21) via a scope connector 29. The light source unit 27 is coupled to the universal cable 23 through a light guide cable 30 via the scope connector 29. The universal cable 23 is configured to include at least a video signal cable through which video signals obtained using the image pickup section are communicated to the image processing unit and a cable (optical fiber) through which illumination light is guided. According to the present embodiment, the universal cable 23 and a control cable 22 extending from the control section 24 are connected to the manipulation section as the integrated cable 3 by being combined together before reaching the manipulation section 3. Of course, the integrated connector connection need not necessarily be used but the units are connected to the manipulation section 3 through the respective cables.

The display unit 25 is configured using, for example, a liquid crystal monitor and can also display a picked-up image and information (for example, position information) on the image in the same screen. The control section 24 includes a motor control section 31 that controllably drives the motor 11. The control section 24 is not limited to a dedicated apparatus but may be configured utilizing a general-purpose processing apparatus, for example, as a personal computer with programs mounted therein.

The insertion section 2 comprises an insertion section main body 4 extending along a longitudinal direction of the insertion section 2, and a bending section 5 provided on a leading end side of the insertion section 2. The insertion section 2 also comprises a rotational driving section 10 provided on the insertion section main body 4 to enable a spiral tube (an introduction propulsion section) 9 to rotate around the longitudinal direction of the insertion section 2, the spiral tube 9 rotating around a longitudinal axis of the insertion section 2 to function as introduction propulsion. In other words, the insertion section main body 4 is formed by connecting the bending section 5, a front flexible hollow shaft 4a, the rotational driving section 10, and a rear flexible hollow shaft 4b together in this order from the leading end side of the insertion section main body 4. A base end portion of the bending section 5 is connected to a front ferrule of the front flexible hollow shaft 4a. A rear ferrule of the front flexible hollow shaft 4a is fitted in and connected to a flange portion of a front cylinder 47 of the rotational driving section 10 as shown in FIG. 2. A front ferrule of the rear flexible hollow shaft 4b is fitted in and connected to an inner circumference of the flange portion of a gear cylinder 51 of the rotational driving section 10. The bending section 5 has a well-known configuration and comprises a plurality of joint rings (not shown in the figure) coupled together so as to be rotationally movable alternately using a lateral direction and a vertical direction as a supporting point. One end of a bending wire (not shown in the figure) is fixed to each of the joint rings. The other end of each bending wire is connected to the knob (not shown in the figure) provided on the manipulation section 3. Operation of the knob allows the bending wires to be pulled in the vertical direction and the lateral direction to tilt and bend the joint rings, to which the bending wires are coupled.

Moreover, a living body introduction apparatus 6 is provided on the insertion section 2.

The living body introduction apparatus 6 comprises a fin formed so as to wrap spirally around the living body introduction apparatus 6 and functioning as a propulsion site (or a backward site). The living body introduction apparatus 6 also comprises the spiral tube (the introduction propulsion section) 9 provided over a casing of the insertion section 2 with a void between the casing and the spiral tube 9 and rotating around the longitudinal axis of the insertion section 2 to function as introduction propulsion. A leading end side of the spiral tube 9 is tapered and thus shaped so as to be easily inserted. The spiral tube 9 can be rotated in both directions (CW and CCW) by the rotational driving section 10. The motor 11, serving as a driving source for the rotational driving section 10, is disposed on the manipulation section 3. One end of a flexible bending shaft 13 is coupled to a rotating shaft of the rotational driving section 10. Furthermore, the shaft 13 penetrates the insertion section 2 and is coupled to one end of a shaft of a driving gear 41 at the other end of the shaft 13 using a coupling member 42.

A rotating force of the motor 11 is transmitted through the flexible bending shaft 13 to rotate the driving gear 41. The coupling member 42 may be configured to allow, during transmission of motor rotation, intervention of a mechanism comprising a plurality of gears for controlling a decrease in the number of rotations and adjusting torque.

The rotating force of the motor 11 is utilized to rotate the spiral tube 9 and rotates the spiral tube 9 in a direction opposite to a spiral direction in which the fin wraps around the living body introduction apparatus to advance the insertion section 2. The spiral tube 9 is removable fitted over the insertion section 2 and comprises a material (for example, a rubber material or a resin material), a structure, or the like which is flexible enough to conform with bending of the bending section 5.

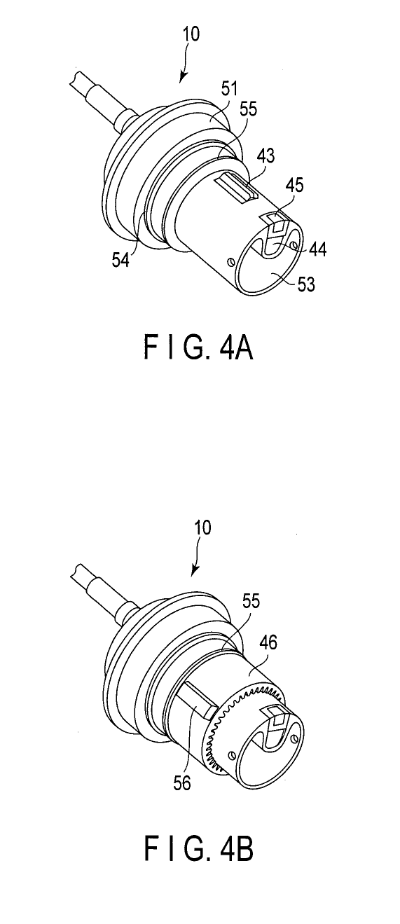

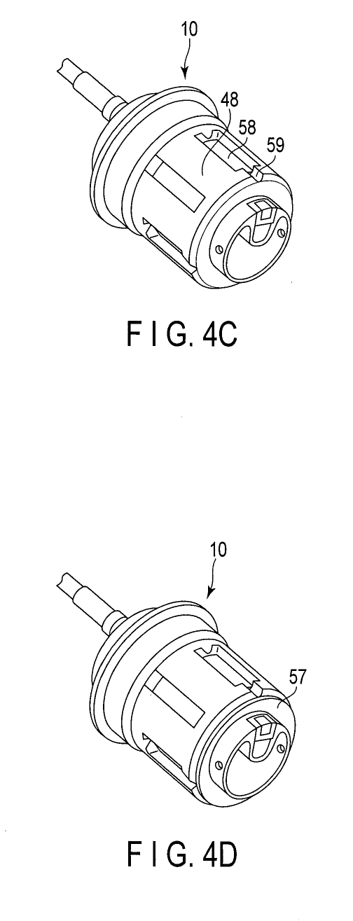

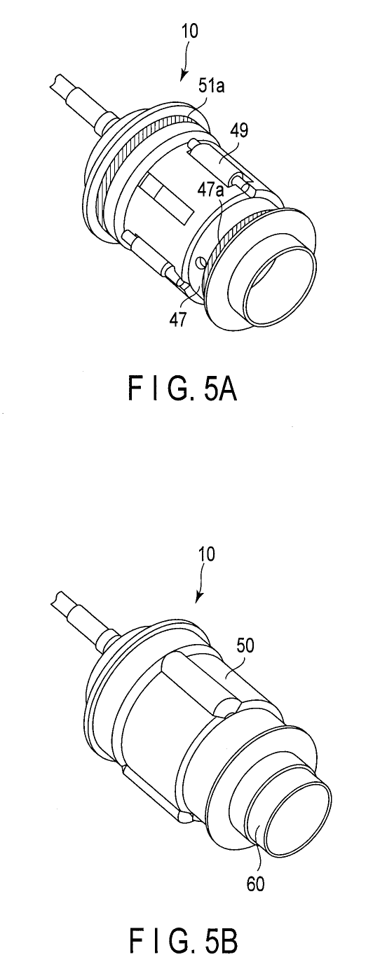

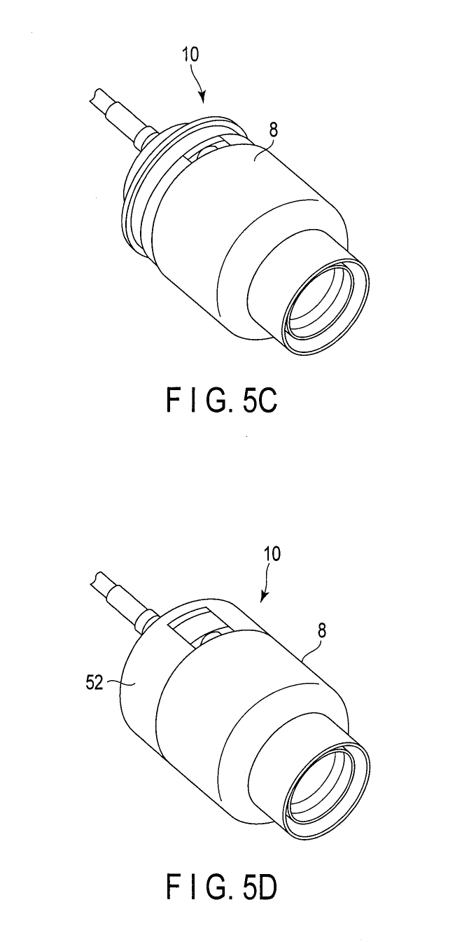

Next, with reference also to FIGS. 4A to 4D and FIGS. 5A to 5D, the structure of the rotational driving section 10 will be described in detail in accordance with an example of an assembly procedure. Here, FIGS. 4A to 4D are diagrams illustrating an assembly structure of the living body introduction apparatus. FIGS. 5A to 5D are diagrams continued from FIG. 4D and illustrating the assembly structure of the living body introduction apparatus. An outer rotating cylinder 8 of the rotating section 10 is configured separately from the spiral tube (the casing) 9, which is the introduction propulsion section.

In a conceptual sense, in the rotating section 10 of the living body introduction apparatus, the driving gear 41 coupled to the shaft 13 meshes with an inner gear 46 serving as a rotating member so that the driving gear 41 is disposed inside an inner rotating cylinder 48 via a transmission gear. The driving gear 41, the transmission gear 43, and the inner gear 46 provide a rotating mechanism. Rotation of the spiral tube 9 not only allows the rotation speed of the motor to be adjusted but also allows the rotation speed and torque to be adjusted by regulating the gear ratio of the above-described gears. The inner rotating cylinder 48 comprises a plurality of rollers rotatable installed on an outer circumferential surface of the inner rotating cylinder 48 and serving as a rotating site. The inner rotating cylinder 48 including the rollers is covered with a cover 50. Opening portions at the opposite ends of the tube-like cover 50 are fixedly bonded to a non-rotating site in a light-tight manner. Moreover, the outer rotating cylinder 8, comprising recesses formed therein and in which the rollers are housed, is fitted over the cover 50.

The living body introduction apparatus will be described below in detail.

As shown in FIG. 2, the driving gear 41 is housed in a gear cylinder 51. A shaft at the other end of the driving gear 41 is fitted in a hole in a retaining cylinder 44 fitted in the gear cylinder 51. The shaft is rotatable and is prevented from slipping out.

Moreover, the retaining cylinder 44 comprises a step formed therein by cutting out a part of an exposed outer circumferential surface side, and a through hole is formed in the step surface so as to extend in a radial direction. A retaining pin 45 is fitted in the through-hole, and a leading end of the pin is fixed in abutting contact with a recessed groove formed in the gear cylinder 51. An upper portion of the inserted retaining pin 45 lies in abutting contact with a rotating shaft of the transmission gear 43 to prevent the transmission gear 43 from slipping out.

As shown in FIG. 4A, an upper portion of the gear of the transmission gear 43 is located to be exposed through the cutout portion of the gear cylinder 51. As shown in FIG. 4B, the annular inner gear 46 is fitted over the gear cylinder 51. The inner gear 46 comprises a gear formed on an inner surface side of the inner gear 46 and meshing with the exposed transmission gear 43. Furthermore, an annular sliding plate 55 is fitted, in an intervening manner, over an inner step surface of the gear cylinder 51 which serves as a sliding surface allowing the gear cylinder 51 and the inner gear 46 to slide with respect to each other when the inner gear 46 is fitted into the gear cylinder 51. Similarly, an annular sliding plate 54 is fitted, in an intervening manner, over an outer step surface of the gear cylinder 51 which serves as a sliding surface allowing the gear cylinder 51 and the outer rotating cylinder 8 to slide with respect to each other when the outer rotating cylinder 8 is fitted over the gear cylinder 51.

Furthermore, the inner gear 46 comprises at least one rectangular key 56 formed in an outer circumferential surface of the inner gear 46. According to the present embodiment, by way of example, the single key 56 disposed on the outer circumferential surface is formed. This configuration allows the transmission gear 43 to rotate in conjunction with rotation of the driving gear 41, in turn rotating the gear of the inner gear 46. Thus, the inner gear 46 rotates on the gear cylinder 51.

Then, as shown in FIG. 4C, the inner rotating cylinder 48, in which three roller storage grooves 58 and a fitting key groove fitted over the key are formed, is fitted over the inner gear 46. In this case, the key 56 is fitted in the key groove.

The key 56 is formed such that a top portion of the key 56 lies as high as or slightly lower than an outer circumferential surface of the inner rotating cylinder 48. Thus, the outer circumferential surface including the top portion of the key 56 is formed to provide a generally smooth surface when the inner rotating cylinder 48 rotates. The rollers 49 are housed in the respective roller storage grooves 58. The rollers 49 are formed of hard metal such as stainless steel. Of course, the present embodiment is not limited to stainless steel, and any hard material is applicable.

As shown in FIG. 2 and FIG. 4C, each of the roller storage grooves 58 comprises two bearing grooves 59 formed at respective opposite ends in a longitudinal direction of the roller storage groove 58 to receive a roller shaft of the roller 49. The bearing grooves 59 are V- or U-shaped and the depth of each of the bearing grooves 59 is designed such that the roller surface of the roller 49 installed in the bearing groove 59 lies slightly away from a surface of the inner gear 46 and is thus free. When, for example, the diameter of the roller 49 is adjusted to synchronize rotation of the inner gear 46 with rotation of the roller, the surface of the inner gear 46 can be brought into contact with a surface of the roller 49 so that rotation of the roller itself helps the roller travel smoothly while passing the cover described below.

Then, as shown in FIG. 4D, the annular sliding plate 57 is fitted over the gear cylinder 51 so as to lie in front of the inner rotating cylinder 48. Moreover, as shown in FIG. 5A, the rollers 49 are installed in the respective roller storage grooves 58. The front cylinder 47 is fitted over the gear cylinder 51 so as to lie in front of the inner rotating cylinder 48, and fixed using screws or the like. The front cylinder 47 comprises a flange portion approximately in the center thereof, and a rear cylindrical portion of the front cylinder 47 is fitted over the gear cylinder 51.

In this configuration, the gear cylinder 51 and the front cylinder 47 are fixed to the gear cylinder 51, and only the inner rotating cylinder 48 located in the center is rotatable.

Then, as shown in FIG. 5B, the cover 50 covers the inner rotating cylinder 48 so that end portions of the cover contact an edge 51a of the gear cylinder 51 and an edge 47a of the front cylinder 47, respectively. As shown in FIG. 5A, the opposite end portions of the cover 50 are fixed in a liquid-tight manner to the edge 51a and the edge 47a, respectively. The liquid-tight fixation is performed using thread winding bonding, an adhesive, a double-sided adhesive tape, or the like.

The cover 50 is formed of a rubber material or a resin material that is non-conductive and stretchable, and prevents a liquid, vapor, or the like from permeating through the cover 50. The cover 50 is annularly shaped. For example, the cover 50 is shaped like a cylinder (a tube) of a size (a diameter) described below. Moreover, annular belts or the like may be fitted on the respective opposite ends of the cover 50 for fixation or reinforcement.

Further, a rear ferrule 60 of the front flexible hollow shaft 4a is fitted into a front cylinder portion of the flange portion of the front cylinder 47 and fixed using screws or the like. In this configuration, the opposite ends of the cover 50 are fixed to the gear cylinder 51 and the front cylinder 47. When the inner rotating cylinder 48, positioned in the center, rotates inside the cover 50, the rollers 49, installed in the inner rotating cylinder 48, rotate while pushing up the cover so that the cover 50 forms protruding portions.

A well-known adhesion inhibitor is sealed inside the cover 50 in order to prevent the abutting contact surfaces of the inner rotating cylinder 48 and the rollers 49 from adhering to an abutting contact surface of the cover 50 (blocking), thus maintaining slippage between the abutting contact surfaces. Moreover, to prevent the cover from being electrically charged (static electricity) in conjunction with rotation of the inner rotating cylinder 48, the members contacted by the cover 50 are desirably at ground potential.

Then, as shown in FIG. 5C, the outer rotating cylinder 8 is fitted over the cover 50, that is, on an outer side of the inner rotating cylinder 48. As shown in section in FIG. 3A, the outer rotating cylinder 8 comprises circular grooves 8a formed at positions corresponding to the rollers 49 arranged in the inner rotating cylinder 48 and each of which has a size sufficient to contain the roller 49 covered with the cover 50. The circular groove 8a is adjusted to form a spacing between the outer rotating cylinder 8 and the cover 50 which serves to prevent the roller 49 covered with the cover 50. The roller 49 is always present, that is, always contained, in the circular groove 8a.

Rotation of the inner rotating cylinder 48 causes the protruding portions of the cover 50 formed by the rollers 49 to push the circular grooves 8a ahead to rotate the outer rotating cylinder 8. Furthermore, as shown in FIG. 5D, a retaining ring 52 is fitted over a base end side of the gear cylinder 51 so as to couple to the outer rotating cylinder 8. Here, the outer rotating cylinder 8 and the retaining ring 52 are coupled together using screws or the like. The surface of the coupled portion is covered with the installed spiral tube 9.

Now, the cover 50 will be described with reference to FIG. 2.

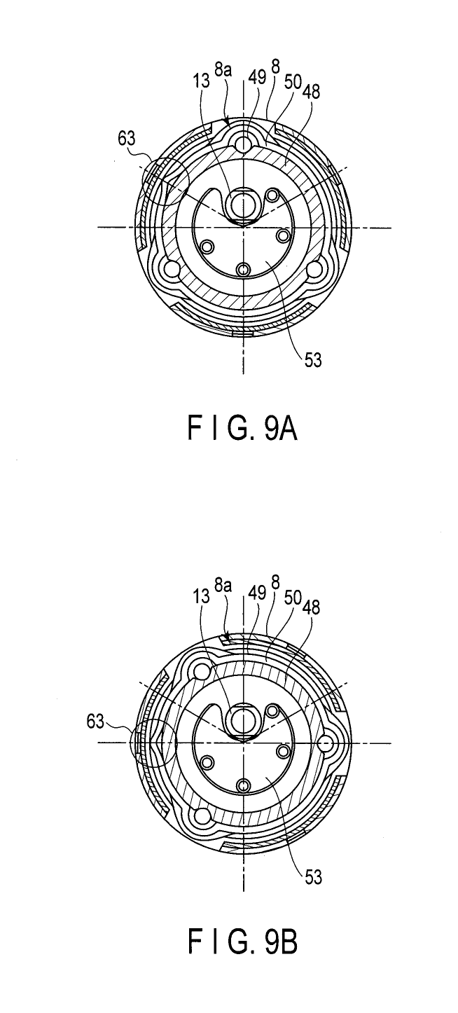

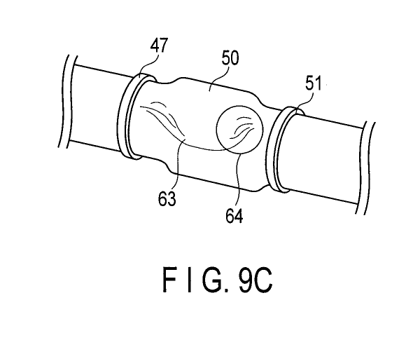

In the above-described pulling caused by slackening or creasing of the cover 50 with the opposite ends thereof fixed. As shown in FIG. 9A, when the cover 50 is subjected to a slack or crease 63, a leading end portion of the slack or crease comes into contact with the inner rotating cylinder and/or the outer rotating cylinder 8. Moreover, when the circular grooves 8a push the slack or crease ahead, the slack or crease moves as shown in FIG. 9B. In conjunction with this movement, a pulling area 64 may be formed in the cover 50 between the gear cylinder 51 and the front cylinder 47, thus damaging the cover 50; for example, tearing the cover 50. Thus, as shown in FIG. 2 and FIG. 5B, when the cover 50 covers the inner rotating cylinder 48 and the rollers 49, the cover 50 needs to contact the inner rotating cylinder 48 with no gap between the cover 50 and the inner rotating cylinder 48 and to be stretched so as to cover the rollers 49 with no gap between the cover 50 and the rollers 49.

Therefore, if the material of the cover 50 is not sufficiently stretchable, when the length of an outer circumference, shown in FIG. 3B, of the inner rotating cylinder 48 with the rollers 49 stored in the roller storage grooves 58 is denoted by an outer circumferential length C, and the length of inner circumference of the cover 50 before installation (covering) is denoted by an inner circumferential length D, the slack or crease 63 can be prevented by establishing a relation “outer circumferential length C inner circumferential length D”. Here, FIG. 3B is a simplified diagram of the cross-sectional view in FIG. 3A showing that the rollers 49 are housed in the inner rotating cylinder 48. The length of a thick line in FIG. 3B is the outer circumferential length C. On the other hand, if the material of the cover 50 is stretchable, when the outer diameter of the inner rotating cylinder 48 is denoted by φA and the inner diameter of the cover 50 before installation (covering) is denoted by φB, the slack or crease 63 can be prevented by establishing a relation “outer circumferential length C>outer circumferential length of inner rotating cylinder 48 π×φA≧inner circumferential length of inner cover 50 π×φB=inner circumferential length D”. The cover 50 stretched under these relations when covering the inner rotating cylinder 48 is in tight contact with the inner rotating cylinder 48. When the rollers 49 rotating on the outer circumferential surface of the inner rotating cylinder 48 come into abutting contact with the cover 50, movement of each of the rollers 49 causes a tensile force to be exerted in a portion positioned in a direction opposite to a rotating direction of the inner rotating cylinder 48 with respect to the roller 46. Thus, the tensile force stretches any slack generated by a push force by the roller 49 in a portion positioned in the rotating direction of the inner rotating cylinder 48, in the direction opposite to the rotating direction. As a result, the slack is prevented from being generated, and maintains the cover 50 in tight contact with the inner rotating cylinder 48.

Then, the protruding portions of the cover 50, formed by the rollers 49, push ahead the respective circular grooves 8a, formed inside the outer rotating cylinder 8, to rotate the outer rotating cylinder 8. The spiral tube 9 is fitted and installed on the outer rotating cylinder 8 to allow the spiral fin 7 with the propulsion function to rotate (forward). Rotating the spiral fin 7 backward (inverse rotation) allows a retraction function to be exerted.

Transmission of the rotational driving force of the whole living body introduction apparatus 6 according to the present embodiment will be described next.

The shaft 13 rotated by the motor 11 rotates the driving gear 41 and further rotates the inner gear 46 via the transmission gear 43, which meshes with the driving gear 41. First, the motor 11, functioning as a driving force generating section generating a rotational driving force rotating the inner gear 46, transmits the rotational driving force to the shaft 13, the driving gear 41, and the transmission gear 43, all of which function as a driving force transmitting section. The rotational driving force is transmitted through the shaft 13 from a base end thereof toward a leading end thereof where the driving gear 41 is provided. The inner rotating cylinder (inner rotor) 48 engages with the key 56, provided on the rotating inner gear 46, and rotates inside the cover 50 along with the inner gear 46. In other words, the inner rotating cylinder (inner rotor) 48 receives the rotational driving force transmitted by the shaft 13, the driving gear 41, and the transmission gear 43, all of which function as a driving force transmitting section, via the inner gear 46, provided on an inner circumferential portion of the inner rotating cylinder (inner rotor) 48 and functioning as a driving force receiving section. The inner rotating cylinder 48 then rotates around the longitudinal axis of the insertion section 2 with respect to the insertion section 2.

In this case, the rollers 49, provided on the outer circumferential surface of the inner rotating cylinder 48, form projecting portions (protruding portions) on the inner rotating cylinder 48. The rollers 49 come into abutting contact with the inner circumference of the cover 50 to push the cover 50 up toward the outside of the inner rotating cylinder 48, thus forming projecting portions (protruding portions) on the cover 50. The projecting portions (protruding portions) formed by the rollers 49 and the cover 50 are fitted into the circular grooves 8a, formed in the outer rotating cylinder (outer rotor) 8, and push an inner wall surface forming circular grooves 61a ahead in conjunction with rotation of the inner rotating cylinder 48. In other words, the outer rotating cylinder 8 comprises engagement sections provided on the inner wall surface of the outer rotating cylinder 8 and engaged by the projecting portions formed by the rollers 49 and the cover 50. The engagement sections are pushed ahead to rotate the outer rotating cylinder 8 along with the inner rotating cylinder 48. Thus, the spiral tube 9 fitted on the outer rotating cylinder 8 is rotated.

As described above, the present embodiment provides a highly resistant liquid-tight structure with a simple configuration and allows the spiral fin disposed on the insertion section to rotate.

Since the cover 50 is equal to or smaller than the inner rotating cylinder 48 in size, the cover 50 avoids rising from the inner rotating cylinder 48 and the rollers 49. This enables prevention of pulling between a fixed area and both the inner rotating cylinder 48 and the outer rotating cylinder 8 and thus prevention of possible damage to the cover, for example, tearing of the cover and the cover peeling off from the fixed area. As a result, a liquid-tight state can be maintained.

Furthermore, the present embodiment provides a configuration in which the rollers 49 push the circular grooves 8a in the outer rotating cylinder 8 ahead via the cover 50 to rotate the outer rotating cylinder 8. The configuration eliminates the need for such small rollers as involved in the conventional technique and is thus simple. This is also effective for reducing costs. Furthermore, omission of structural members further improves the effect of sterilization.

Since the outer rotating cylinder 8 is separate from the spiral tube 9, the spiral tube 9 can be selectively installed which comprises a fin suitable for a lumen to be observed. Here, when an inner circumferential surface of the spiral tube 9 and an outer circumferential surface of the outer rotating cylinder 8 are shaped like a polygon such as an octagon and formed to be engageable with each other, the spiral tube 9 with the insertion section 2 inserted along a longitudinal axis of the spiral tube 9 can be fixed to the outer circumferential surface of the outer rotating cylinder 8. Furthermore, replacement only of the spiral tube 9 that is expandable allows a reduction in costs.

### Second Embodiment

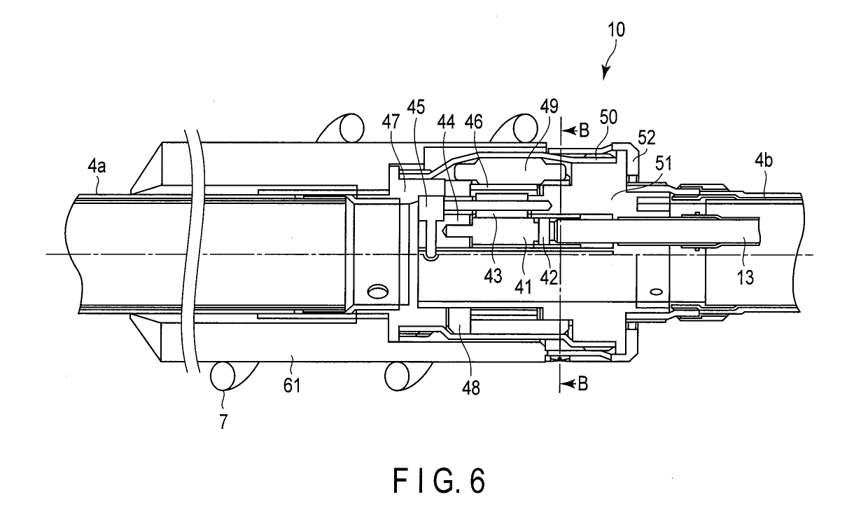

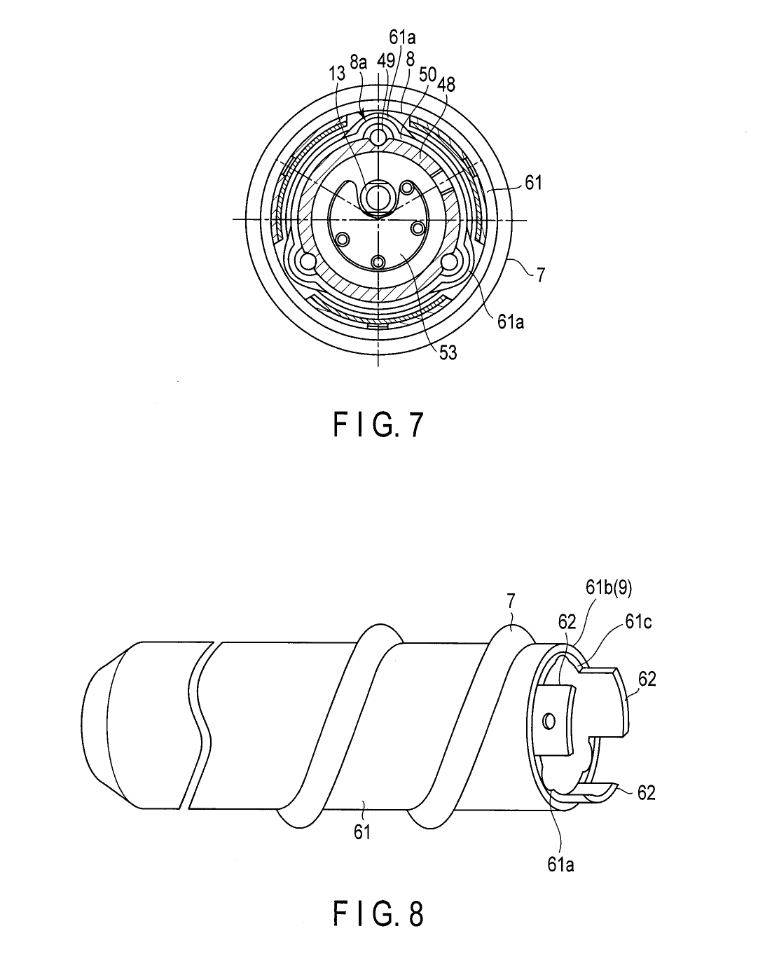

FIG. 6 is a diagram showing a configuration of a living body introduction apparatus according to a second embodiment in section. FIG. 7 is a cross-sectional view showing the configuration and taken along B-B in FIG. 6. FIG. 8 is a diagram showing an external configuration of an outer rotating cylinder configured integrally with a spiral tube (a casing) of the living body introduction apparatus. Structural sites of the present embodiment which are equivalent to corresponding structural sites of the first embodiment are denoted by the same reference numerals.

In an endoscope apparatus 1 according to the present embodiment, a manipulation section and a driving unit system are configured equivalently to the manipulation section and driving unit system according to the above-described first embodiment, respectively. The present embodiment differs from the first embodiment in that a spiral tube 9 and an outer rotating cylinder 8 of a living body introduction apparatus 6 on an insertion section 2 are integrated together. Here, the description of the manipulation section and the driving unit system is omitted, and the living body introduction apparatus 6 will be described.

In a conceptual sense, in the living body introduction apparatus 6 according to the present embodiment, a driving gear 41 coupled to a shaft 13 meshes, via a transmission gear 43, with an inner gear 46 disposed inside an inner rotating cylinder 48. A plurality of rollers are rotatably installed on the inner rotating cylinder 48. The inner rotating cylinder 48 including the rollers is covered with a cover 50. Opposite ends of the cover 50 are fixedly bonded to non-rotating sites in a liquid-tight manner. The above-described configuration is equivalent to the corresponding configuration of the above-described first embodiment.

As is the case with the first embodiment, the cover 50 covers the inner rotating cylinder so that opposite end portions of the cover contact a gear cylinder 51 and a front cylinder 47. The cover 50 is fixed in a liquid-tight manner using thread winding bonding, an adhesive, a double-sided adhesive tape, or the like.

Thus, if the material of the cover 50 is not sufficiently stretchable, when the length of an outer circumference of the inner rotating cylinder 48 with rollers 49 stored in roller storage grooves 58 is denoted by an outer circumferential length C as shown in FIG. 3B, and the length of inner circumference of the cover 50 before installation (covering) is denoted by an inner circumferential length D, a possible slack or crease 63 can be prevented by establishing a relation “outer circumferential length C inner circumferential length D”. On the other hand, if the material of the cover 50 is stretchable, when the outer diameter of the inner rotating cylinder 48 is denoted by φA and the inner diameter of the cover 50 before installation (covering) is denoted by φB, the slack or crease 63 can be prevented by establishing a relation “outer circumferential length C>outer circumferential length of inner rotating cylinder 48 π×φA≧inner circumferential length of inner cover 50 π×φB=inner circumferential length D”.

An outer rotating cylinder 61 according to the present embodiment is configured such that a spiral tube with a spiral fin formed thereon is integrally mounted on an outer circumferential surface of the outer rotating cylinder 61. The outer rotating cylinder 61 is fitted over the cover 50.

As shown in FIG. 7, a base end-side opening portion of the outer rotating cylinder 61 fitted over the inner rotating cylinder 48 including the rollers has a two-layer structure with a soft spiral tube portion 61b and a metal portion or hard resin portion (the portion corresponding to the outer rotating cylinder 8 according to the first embodiment) 61C. Recesses are formed on an inner surface side of the hard portion so as to house the rollers 49. Each of the recesses is shaped like a circle, a rectangle, or the like so to be able to house the roller 49.

Furthermore, as shown in FIG. 8, the outer rotating cylinder 61 comprises a plurality of mounting sections 62 provided at a base end-side opening end of the hard portion and extending in a longitudinal direction so as to couple to a retaining ring 52. The mounting sections 62 and the retaining ring 52 are coupled together using screws or the like. Coupling portions including the screws are covered with insulating partial covers (not shown in the figures). The outer rotating cylinder 61 is fitted over the inner rotating cylinder 48 via the cover 50 so that the rollers 49 covered with the cover 50 are contained in the recesses as shown in FIG. 6 and FIG. 7. Each of the recesses is shaped like a circle, a rectangle, or the like so to be able to house the roller 49. A case where circular grooves 61a are formed as the recesses will be described below.

In the living body introduction apparatus 6 according to the preset embodiment, the shaft 13 rotated by a motor 11 rotates the inner gear 46 via the transmission gear 43, which meshes with and rotates the driving gear 41. First, the motor 11, functioning as a driving force generating section generating a rotational driving force rotating the inner gear 46, transmits the rotational driving force to the shaft 13, the driving gear 41, and the transmission gear 43, all of which function as a driving force transmitting section. The rotational driving force is transmitted through the shaft 13 from a base end thereof toward a leading end thereof where the driving gear 41 is provided. The rotational driving force is then transmitted to the inner gear 46 via the driving gear 41 and the transmission gear 43. The inner rotating cylinder (inner rotor) 48 engages with a key 56 provided on the rotating inner gear 46 and rotates inside the cover 50 along with the inner gear 46. In other words, the inner rotating cylinder (inner rotor) 48 receives the rotational driving force transmitted by the shaft 13, the driving gear 41, and the transmission gear 43, all of which function as a driving force transmitting section, via the inner gear 46, provided on an inner circumferential portion of the inner rotating cylinder (inner rotor) 48 and functioning as a driving force receiving section. The inner rotating cylinder 48 then rotates around a longitudinal axis of the insertion section 2 with respect to the insertion section 2.

In this case, the rollers 49, provided on the outer circumferential surface of the inner rotating cylinder 48, form projecting portions (protruding portions) on the inner rotating cylinder 48. The rollers 49 come into abutting contact with the inner circumference of the cover 50 to push the cover 50 up toward the outside of the inner rotating cylinder 48, thus forming projecting portions (protruding portions) on the cover 50. The projecting portions (protruding portions) formed by the rollers 49 and the cover 50 are fitted into the circular grooves 61a, formed in the outer rotating cylinder 61 (outer rotor), and push an inner wall surface forming the circular grooves 61a ahead in conjunction with rotation of the inner rotating cylinder 48. In other words, the outer rotating cylinder 61 (outer rotor) comprises engagement sections provided on the inner wall surface of the outer rotating cylinder 61 (outer rotor) and engaged by the projecting portions formed by the rollers 49 and the cover 50. The engagement sections are pushed ahead to rotate the outer rotating cylinder 61 (outer rotor) along with the inner rotating cylinder 48.

As described above, the present embodiment is effective for reducing costs using a simple configuration, provides a highly resistant liquid-tight structure, and allows the spiral fin disposed on the insertion section to rotate. Since the cover 50 is equal to or smaller than the inner rotating cylinder 48 in size as is the case with the above-described first embodiment, the cover can be prevented from being damaged, for example, being torn and from being peeled off from a fixed area. As a result, a liquid-tight state can be maintained. Furthermore, the outer rotating cylinder integrated with the spiral tube is formed to be inserted through the insertion section 2 along the longitudinal axis of the insertion section 2. The outer rotating cylinder is further formed such that the projecting portions (protruding portions) formed by the cover 50 and the rollers 49 are removably fitted into the recesses (circular grooves or engagement sections) in the outer rotating cylinder. Thus, the outer rotating cylinder can be easily installed and removed.

The above-described embodiments include the following features of the invention.

[1] A living body introduction apparatus comprising:

an insertion section comprising a longitudinal axis;

a rotational moving member configured to rotatably move the insertion section 2 around the longitudinal axis;

a rotational moving holding section configured to hold the rotational moving section in such a manner that the rotational moving member is rotationally movable with respect to the insertion section;

a rotating cylinder provided inside the insertion section and configured to be rotatable around the longitudinal axis;

a rotating mechanism configured to rotate the rotating cylinder;

a covering member comprising opposite end portions and formed like a cylinder in such a manner that an inner circumferential length of the covering member before mounting on the insertion section is set equal to or smaller than an outer circumferential length of the rotating cylinder, the covering member covering the rotating cylinder, an inner circumference of the opposite ends being fixed to an outer circumference of the insertion section; and

an engagement member disposed at an outer circumferential position on the rotating cylinder and projecting in an outer circumferential direction of the rotating cylinder to deform the covering member in the outer circumferential direction, the engagement member coming into engagement with the rotational moving member via the covering member in response to rotational movement of the rotating cylinder and being able to rotationally move the rotational moving member.

[2] The living body introduction apparatus set forth in [1], wherein a plurality of the engagement members are disposed on a circumference of the rotating cylinder.

[3] The living body introduction apparatus set forth in [2], wherein the engagement members are disposed rotationally symmetrically with respect to the longitudinal axis.

[4] The living body introduction apparatus set forth in [1], wherein the engagement members are cylindrical rollers configured to be rotationally movable with respect to the covering member along with the rotating cylinder.

[5] The living body introduction apparatus set forth in [4], wherein the rollers are slidable with respect to the rotating cylinder.

[6] The living body introduction apparatus set forth in [1], wherein the covering member keeps an inside of the insertion section liquid-tight.

[7] The living body introduction apparatus set forth in [1], wherein the covering member is stretchable.

[8] The living body introduction apparatus set forth in [7], wherein an inner diameter of the covering member before mounting on the insertion section is equal to or smaller than an outer diameter of the rotating cylinder.

[9] The living body introduction apparatus set forth in [1], wherein the rotational moving member comprises a spiral fin section along the longitudinal axis.

[10] The living body introduction apparatus set forth in [9], wherein the rotational moving member is provided in an intermediate portion of the insertion section.

[11] The living body introduction apparatus set forth in [10], wherein the insertion section comprises a bending section at a position closer to a leading end than the intermediate portion.

[12] The living body introduction apparatus set forth in [1], wherein the rotating mechanism comprises a shaft comprising a longitudinal axis and configured to be bendable enough to transmit rotational movement from a base end side to a leading end side of the insertion section.

[13] The living body introduction apparatus set forth in [12], wherein the rotating mechanism comprises a gear unit configured to transmit the rotational movement from a leading end of the shaft to the rotating cylinder so as to rotationally move the rotating cylinder.

[14] The living body introduction apparatus set forth in [13], wherein the rotating cylinder comprises a circumferential gear provided on an inner circumferential surface and to which the rotational movement is transmitted by the gear unit.

1. An introduction apparatus comprising:
an insertion section configured to extend along a longitudinal axis and to be able to be inserted into a lumen;
a rotational driving source which generates rotational driving force;
an inner rotating cylinder provided in the insertion section at any position on the insertion section and annularly configured to be rotatable around the longitudinal axis, the inner rotating cylinder having a projecting portion on an outer circumferential surface and rotating in accordance with the rotational driving force from the rotational driving source;
a covering member configured to have an inner circumferential length equal to or smaller than an outer circumferential length of the inner rotating cylinder including the projecting portion, the covering member annularly covering the inner rotating cylinder including the projecting portion in watertight contact with the inner rotating cylinder, and the covering member fixing opening ends thereof at opposite ends thereof to non-rotating sites located beyond the inner rotating cylinder;
an outer rotating cylinder comprising an engagement section which is formed on an inner circumferential side thereof and which is engageable with the projecting portion covered with the covering member, the outer rotating cylinder being fitted on a casing side of the inner rotating cylinder covered with the covering member, the engagement section being pushed ahead and rotated by a protruding portion of the covering member pushed up by the projecting portion as a result of rotation of the inner rotating cylinder; and
an introduction propulsion unit rotatable along with the outer rotating cylinder and comprising a spiral propulsion site on an outer surface of the introduction propulsion unit, the introduction propulsion unit supporting rotational introduction into the lumen.

2. The introduction apparatus according to claim 1, wherein the projecting portion comprises a rotating site.

3. The introduction apparatus according to claim 1, wherein the covering member is stretchable and has an inner diameter equal to or smaller than an outer diameter of the inner rotating cylinder.

4. The introduction apparatus according to claim 1, wherein a plurality of rotating sites are disposed rotationally symmetrically with respect to the longitudinal axis and configured to move rotationally on the covering member between the rotating inner rotating cylinder and the fixed covering member.

5. The introduction apparatus according to claim 2, wherein the rotating site is a cylindrical roller which is rotationally movable with respect to the inner rotating cylinder and the covering member.

6. The introduction apparatus according to claim 1, wherein the propulsion site is spirally formed along the longitudinal axis of the outer rotating cylinder.

7. The introduction apparatus according to claim 1, wherein the insertion section comprises a bending section at a position closer to a leading end than the introduction propulsion unit.

8. The introduction apparatus according to claim 1, comprising a flexible shaft configured to couple the rotational driving source to the inner rotating cylinder through the insertion section to transmit a rotating force.

9. The introduction apparatus according to claim 1, comprising a rotating mechanism disposed between the inner rotating cylinder and the shaft and comprising a plurality of gears configured to convert rotation from the rotational driving source into any rotation speed and a rotating force.

10. An introduction propulsion unit configured to be detachably attached to an endoscope, the introduction propulsion unit comprising:
an insertion section configured to extend along a longitudinal axis and to be able to be inserted into a lumen;
a rotational driving source which generates rotational driving force;
an inner rotating cylinder provided in the insertion section at any position on the insertion section and annularly configured to be rotatable around the longitudinal axis, the inner rotating cylinder having a projecting portion on an outer circumferential surface and rotating in accordance with the rotational driving force from the rotational driving source; and
a covering member configured to have an inner circumferential length equal to or smaller than an outer circumferential length of the inner rotating cylinder including the projecting portion, the covering member annularly covering the inner rotating cylinder including the projecting portion in watertight contact with the inner rotating cylinder, and the covering member fixing opening ends thereof at opposite ends thereof to non-rotating sites located beyond the inner rotating cylinder;
the introduction propulsion unit comprising an outer rotating cylinder comprising an engagement section which is formed on an inner circumferential side thereof and which is engageable with the projecting portion covered with the covering member, the outer rotating cylinder being fitted on a casing side of the inner rotating cylinder covered with the covering member, the engagement section being pushed ahead and rotated by a protruding portion of the covering member pushed up by the projecting portion as a result of rotation of the inner rotating cylinder, the introduction propulsion unit being rotatable along with the outer rotating cylinder and comprising a spiral propulsion site on an outer surface of the introduction propulsion unit, the introduction propulsion unit supporting rotational introduction into the lumen.

11. An endoscope comprising:
an insertion section configured to extend along a longitudinal axis and to be able to be inserted into a lumen;
a rotational driving source which generates rotational driving force;
an inner rotating cylinder provided in the insertion section at any position on the insertion section and annularly configured to be rotatable around the longitudinal axis, the inner rotating cylinder having a projecting portion on an outer circumferential surface and rotating in accordance with a rotational driving force from the rotational driving source; and
a covering member configured to have an inner circumferential length equal to or smaller than an outer circumferential length of the inner rotating cylinder including the projecting portion provided on the outer circumferential surface of the inner rotating cylinder, the covering member annularly covering the inner rotating cylinder including the projecting portion in watertight contact with the inner rotating cylinder, the covering member fixing opening ends thereof at opposite ends thereof to non-rotating sites located beyond the inner rotating cylinder, the covering member comprising an outer rotating cylinder comprising an engagement section formed on an inner circumferential side thereof and which is engageable with the rotating site covered with the covering member, the outer rotating cylinder being fitted on a casing side of the inner rotating cylinder covered with the covering member, the engagement section being pushed ahead and rotated by a protruding portion of the covering member pushed up by the projecting portion as a result of rotation of the inner rotating cylinder, an introduction propulsion unit being able to be installed on the covering member and being fitted over the outer rotating cylinder in such a manner as to cover an outer surface of the outer rotating cylinder and comprising a spiral propulsion site on an outer surface of the introduction propulsion unit, the introduction propulsion unit supporting rotational introduction into the lumen.
